(19) United States
(12) Patent Application Publication
(10) Pub. No.: US 2010/0193698 A1
(43) Pub. Date: Aug. 5, 2010

(54) GANTRY MOUNTED PATIENT TABLE AND EXCHANGER FOR MEDICAL IMAGING

(75) Inventor: Rizwan S. Hassan, Danville, CA (US)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 11/913,848

(22) PCT Filed: May 9, 2006
(86) PCT No.: PCT/IB06/51453

§ 371 Date: Nov. 8, 2007

Related U.S. Application Data
(60) Provisional application No. 60/681,367, filed on May 16, 2005.

(51) Int. Cl. A61B 6/04; G01T 1/161; B23P 17/04

(52) U.S. Cl. 250/394; 250/491.1; 250/395; 29/428

(57) ABSTRACT

The following relates to an integrated patient scanner system. The system includes a stationary gantry with first and second sides and a rotating gantry mounted to rotate in the stationary gantry around a patient receiving aperture, which is defined by the rotating gantry. The system further includes at least one detector mounted to the rotating gantry and extending from the first side around the patient receiving aperture. A patient table having a pedestal and a pallet is mounted to the stationary gantry. The pedestal mounts to and extends from the second side of the stationary gantry opposite to the at least one detector such that the pedestal and the stationary gantry define a common T-shaped supporting surface adapted to rest on the floor. The patient pallet mounts to the pedestal for movement to extend cantilevered through the patient aperture and out the first side past the at least one detector.

The following generally relates to medical imaging arts. It finds particular application to Single Photon Emission Computed Tomography (SPECT) systems and is best described with respect to a SPECT system that includes a stationary and rotating gantry, at least one detector, a patient table, and a collimator exchange and storage system. However, it is to be appreciated that some aspects are also applicable to other imaging modalities.

SPECT imaging systems typically utilize one or more detectors (Gamma cameras) that are either mounted on a rotating gantry or robotic arms. Gantry-mounted detectors rotate with gantry componentry located within a stationary gantry in order to rotate around a patient that is variously positioned, via a patient table, within an imaging region between the one or more detectors. The patient table includes a patient pallet and a pedestal that houses associated electrical, mechanical and/or software components. Typically, the pallet cantilevers the patient into a scanning position. The patient lies on the pallet, which has an arced and narrow configuration that conforms to the patient's body and allows the detectors to be variously positioned with respect to the patient, including in close proximity to the patient.

The patient table, the gantry, a user console, a collimator exchanger and storage system, etc. are shipped separately and aligned on site. The floor, typically concrete, is tapped to receive mounting bolts for the various components. The gantry and the patient table are separately mounted to the floor and then aligned through a detailed and labor-intensive precision alignment procedure.

With some systems, the gantry translates linearly along the table axis on tracks that are mounted on the floor. These tracks require shimming or other adjustment to compensate for the floor flatness variations. Floor flatness compensating schemes can be time consuming and may require periodic adjustment.

In some systems, the patient table is released from its floor mounts and rolled on casters. With these systems, the patient table is manually maneuvered out of the way when imaging a patient from a hospital bed or wheelchair, and later reattached to the floor mounts.

The collimator exchanger and storage device is also shipped separately from the gantry and, thus, has to be assembled and calibrated with the gantry on site. Typically, the collimator storage rack is a separate structure that is mounted along a wall or other out-of-the-way place. A collimator transport cart is used to move collimators between the storage rack and the detector heads.

Gantries with two scintillation cameras (e.g., duel head systems) typically provide a clinician with an option of conducting SPECT studies with the detectors in at least two configurations: in one configuration, the heads are at about 180 degrees apart from each other while they rotate around the patient; and in the other configuration, the heads are approximately 90 degrees apart from each other while they rotate around the patient. These two configurations accommodate conventional and cardiac SPECT studies. Some systems provide one or more additional angles (e.g., 101 degrees). This limited number of configurations (e.g., 90, 101 and 180 degrees) prohibits technicians (users) from adjusting relative angles between two heads in order to optimize images. Due to such limited configurations, some conventional systems are insufficiently versatile, inconvenient to use, and/or not suitable for certain types of procedures such as studies in which the patient stands or sits. Those systems with heads that are circumferentially adjustable to a larger array of positions typically include additional motors, mechanical systems, and other complexities.

The following relates to an integrated patient scanner system. The system includes a stationary gantry with first and second sides and a rotating gantry mounted to rotate in the stationary gantry around a patient receiving aperture, which is defined by the rotating gantry. The system further includes at least one detector mounted to the rotating gantry and extending from the first side around the patient receiving aperture. A patient table having a pedestal and a pallet is mounted to the stationary gantry. The pedestal mounts to and extends from the second side of the stationary gantry opposite to the at least one detector such that the pedestal and the stationary gantry define a common T-shaped supporting surface adapted to rest on the floor. The patient pallet mounts to the pedestal for movement to extend cantilevered through the patient aperture and out the first side past the at least one detector.

An advantage includes pre-calibration of the patient table with the gantry and mitigating need for on-site patient table-to-gantry calibration.

In another advantage, the patient table pedestal serves as a counter weight to the patient on a cantilevered pallet.

Another advantage resides in reduced time and cost associated with assembling and setting up a system.

Another advantage is integrating the patient table with the back of the gantry.

Another advantage is opening access to the front of the scanner.

Another advantage resides in eliminating the floor as the alignment reference which floor can vary in flatness and cause misalignment between the detector head and the patient.

Another advantage resides in a reduced footprint.

Another advantage is the ability to configure a two head system at an increased (essentially infinite) number of detector relative angles between a range of about 90 degrees to about 180 degrees, without additional drive motors.

Another advantage is integrating a collimator exchange and storage mechanism with the gantry.

Another advantage is a collimator exchanger that is easy to use and consumes minimal usable floor space.

Still further advantages will become apparent to those of ordinary skill in the art upon reading and understanding the detailed description of the preferred embodiments.

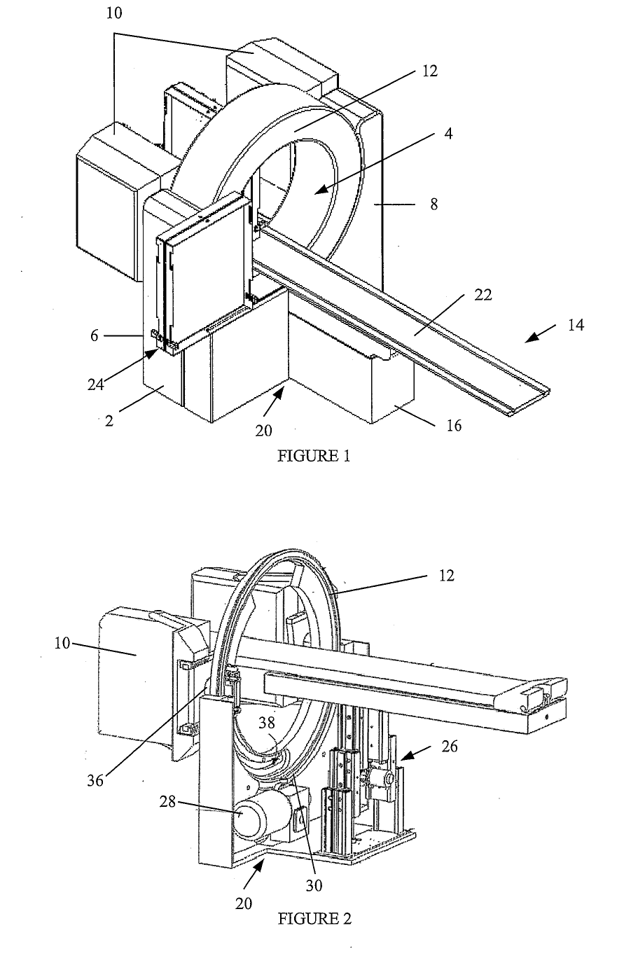

FIG. 1 illustrates an integrated patient scanning system used for medical imaging applications.

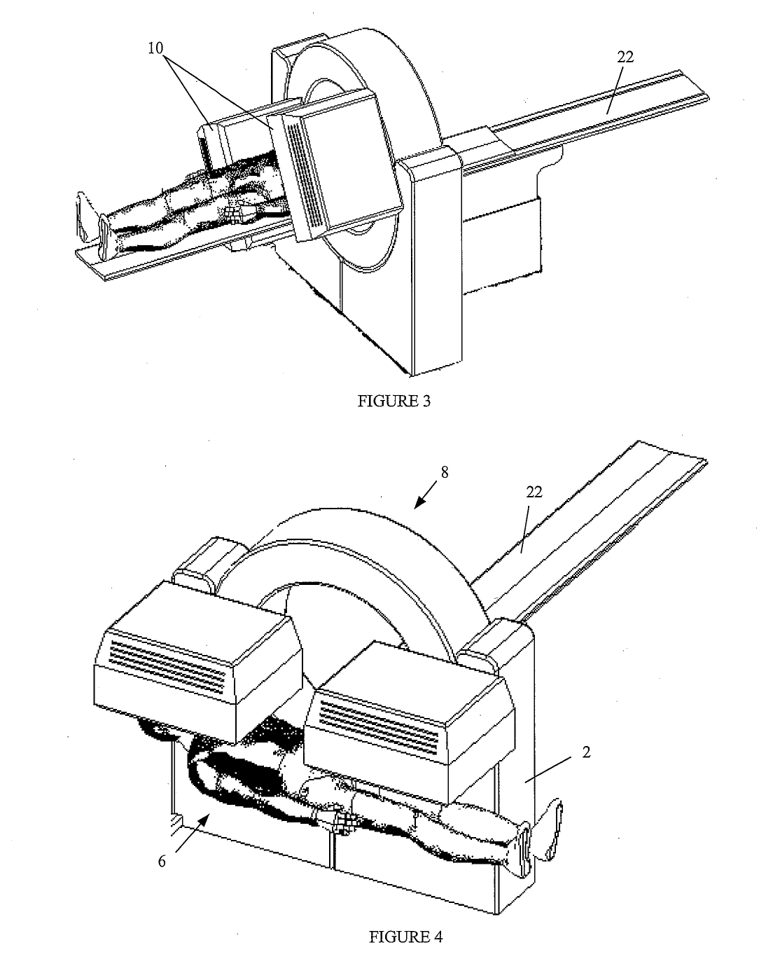

FIG. 2 illustrates an exemplary technique for connecting the patient table and the stationary gantry to form a single unit.

FIG. 3 shows the detector heads positioned in a 180 degree configuration.

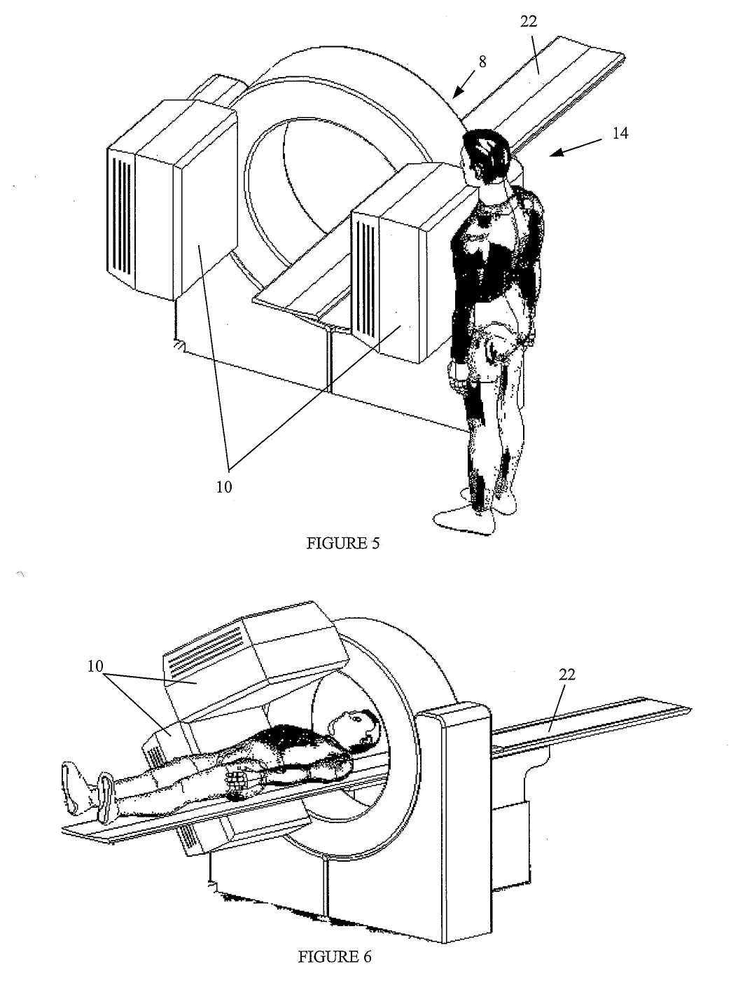

FIG. 4 shows the pallet retracted behind the stationary gantry and the patient supported by an ancillary for scanning.

FIG. 5 provides an example of imaging a standing patient with the patient pallet retracted behind the stationary gantry.

FIG. 6 shows the detector heads positioned in a 90 degree configuration.

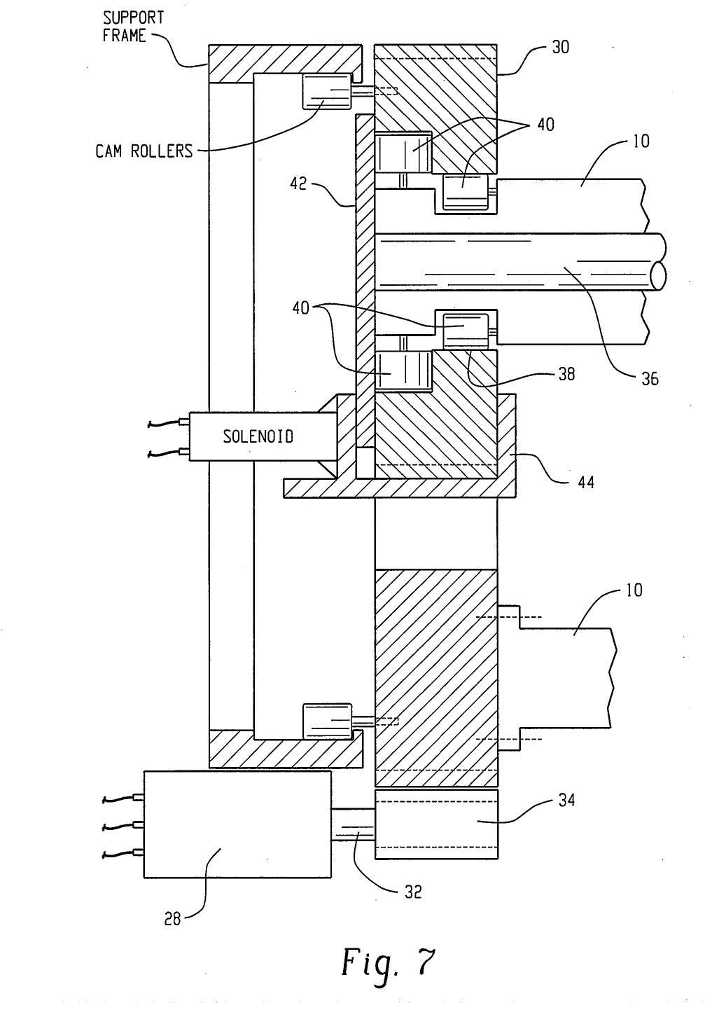

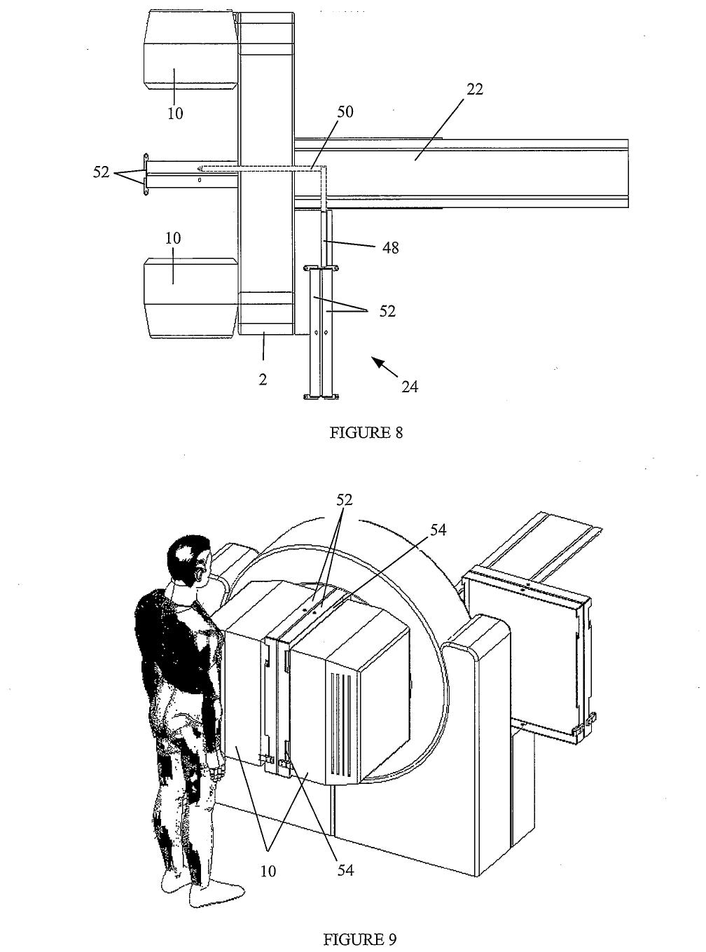

FIG. 7 illustrates an exemplary technique to positioning detectors in a relative angle between about 90 and 180 degrees.

FIG. 8 illustrates a top-down view of the collimator exchange and storage system mounted to the stationary gantry.

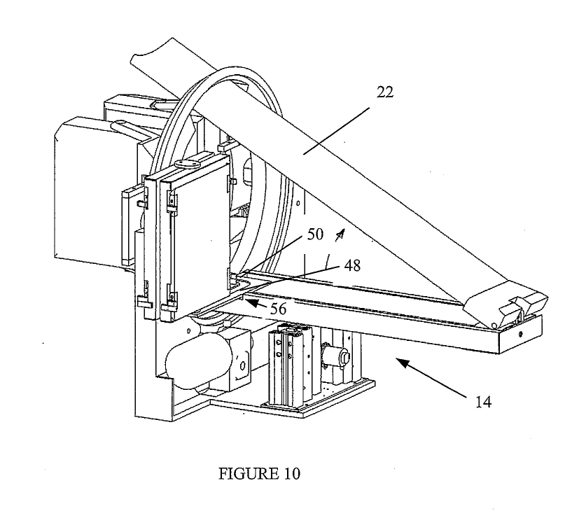

FIG. 9 illustrates the detectors drawn to the collimator exchange and storage system.

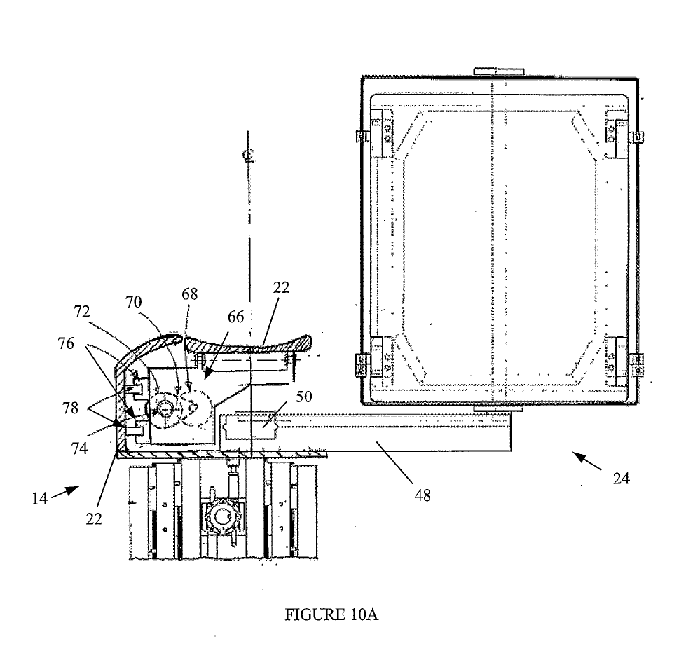

FIG. 10 shows the location of the delivery track and the extension track of the collimator exchange and storage system with respect to the patient table.

FIG. 10a illustrates a location of the collimator exchange and storage system with respect to a table horizontal drive mechanism of a patient table.

FIG. 11 shows an assembly with the delivery track and extension track.

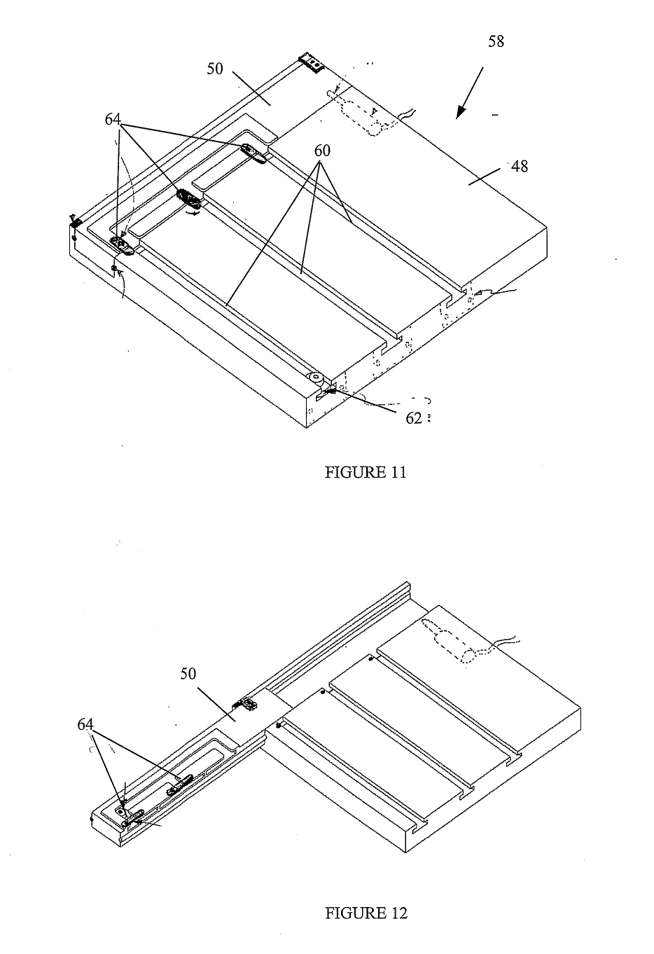

FIG. 12 illustrates an extended extension track with latched spring safety latches.

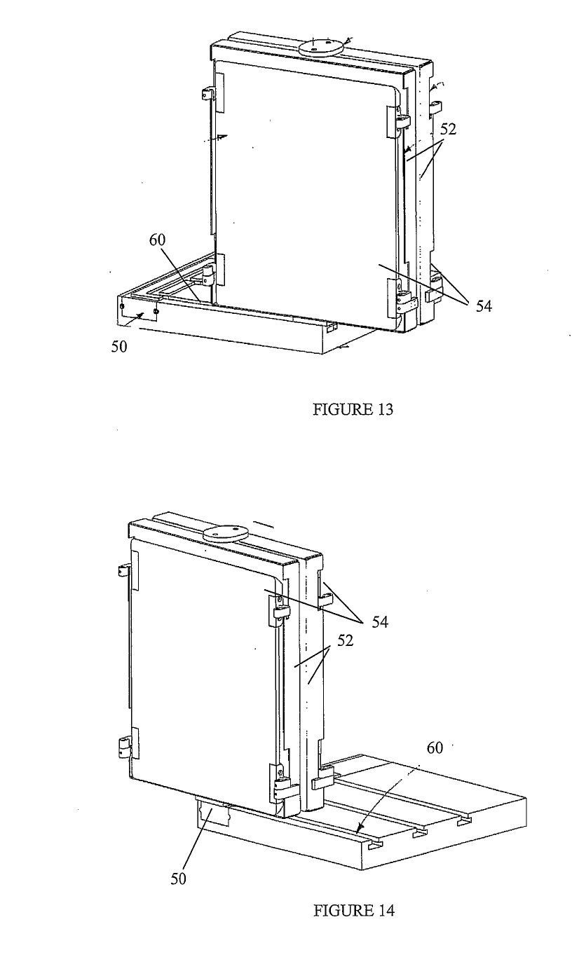

FIG. 13 shows gimbals with collimators in a storage position in the delivery track.

FIG. 14 shows the gimbals partially moved along the delivery track.

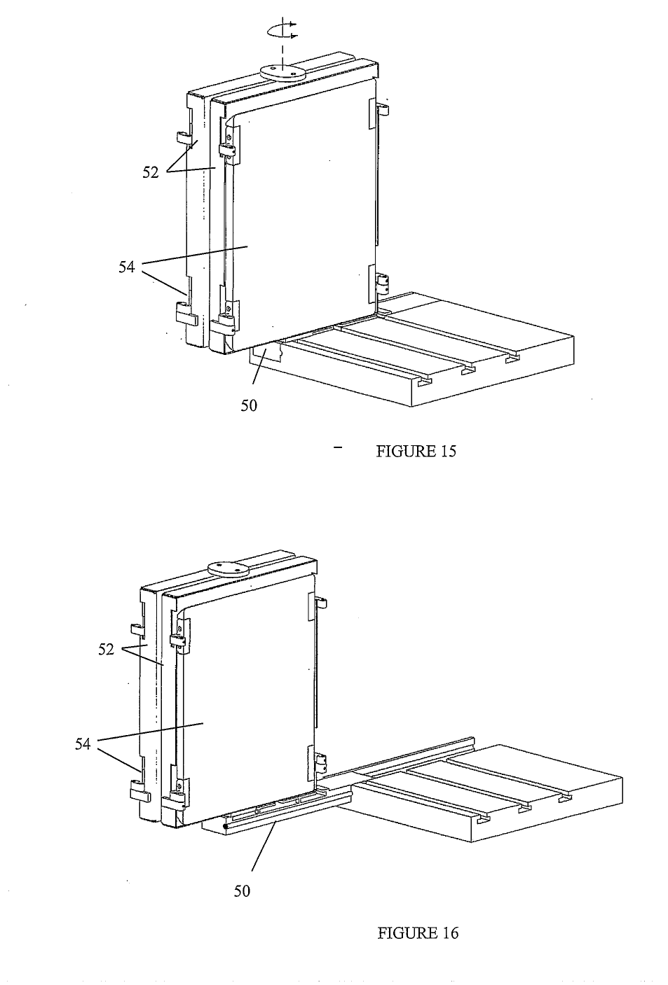

FIG. 15 shows gimbals rotated about a pivot onto the extension track.

FIG. 16 shows the gimbals extended on the extension track to a position proximate the detectors for loading/unloading collimators.

FIG. 1 illustrates an integrated patient scanning system used for medical imaging applications (e.g., diagnostic) such as, for example, Single Photon Emission Computed Tomography (SPECT), Positron Emission Tomography (PET), and Computed Tomography (CT). The system includes a stationary gantry 2 that defines a first face or side 6 and a second face or side 8. The system further includes a rotating gantry 12 rotatably mounted to rotate in the stationary gantry 2 and which defines an aperture 4. At least one detector head 10 is mounted to the rotating gantry and extends from the first face 6 around and on the first side 6 of the patient receiving aperture 4. It is to be appreciated that although a two detector head configuration is shown in FIG. 1, essentially any number of detectors (e.g., one, three . . . ) can be utilized in accordance with various embodiments. The system further includes a patient table 14 having a pedestal 16 mounted to and extending from the second side 8 of the stationary gantry 2 opposite to the at least one detector 10 such that the pedestal 16 and the stationary gantry 2 define a T-shaped lower supporting surface 20 adapted to rest on the floor. The patient table 14 further includes a patient pallet 22 mounted to the pedestal 16 and extending cantilevered through the patient aperture 4 and out the first side 6 past the at least one detector 10. The patient pedestal 16 is mounted to the gantry 2 such that it does not interfere with the at least one detector 10 on the first side 6 of the patient receiving aperture 4.

The pallet 22 supports a patient during scanning or patient imaging. For example, the patient lies (e.g., prone, supine, etc.) on the pallet, which can then be suitably positioned with respect to the at least one detector 10. In some instances, the pallet 22 is fully retracted to the second side 8 of the patient receiving aperture 4 and the patient stands, sits, or lies, for example, on another device (not shown) such as a gurney, a bed, etc. The system also includes a collimator exchange and storage system 24 mounted to the second side 8 of the stationary gantry 2. The collimator exchange and storage system 24 stores one or more sets of collimators and facilitates loading/unloading a set of collimators from the at least one detector 10.

The system mitigates calibration and interferences issues associated with conventional systems by factory aligning the patient table 14 and the stationary gantry 2 through the common T-shaped supporting surface 20 on the second side 8 opposite the at least one detector 10 to form a single unit. The factory aligned pedestal 16 and the stationary gantry 2 are then shipped as a single integrated unit and installed at its destination as a pre-calibrated unit. This factory alignment eliminates on-site patient table-to-stationary gantry alignment and intermediary electrical and mechanical connections. Since the patient table 14 is mounted to the stationary gantry 2 on the second side 8 opposite the at least one detector 10, the pedestal 16 does not interfere with the imaging region and the pallet 22 can be retracted to remove it from the imaging region. Preferably, the pedestal 16 is anchored to the floor at its outer end to act as a lever arm to counter the forces attributable to the detector heads 10 and the patient cantilevered to the first side 6.

Furthermore, with this single unit configuration the stationary gantry 2 is utilized as a frame of reference (rather than the floor) for the patient table 14. For instance, the patient table 14 is factory calibrated to a coordinate system or space defined by the stationary gantry 2. This frame of reference is used when driving the patient table through a vertical motion and a horizontal motion.

Similar to the patient table 14, the collimator exchange and storage system 24 is mounted to the stationary gantry 2. This provides factory calibration of the collimator exchange and storage system 24 with the stationary gantry 2. The collimator exchange and storage system 24 and stationary gantry 2 is then shipped to its destination and assembled as a single unit without need for on-site collimator exchange and storage system-to-stationary gantry calibration. Even if the pedestal 16 or the exchange and storage system 24 are disconnected from the stationary gantry 2, e.g., to move the components through a narrow doorway, they are readily reconnected in their factory aligned relative positions.

FIG. 2 illustrates an exemplary technique for connecting the patient table 14 and the stationary gantry 2 to form a single unit. The T-shaped supporting surface 20 is used as a base plate and plane of reference for both the stationary gantry 2 and the patient table 14. The T-shaped supporting surface 20 includes a portion that is formed to accommodate a vertical drive assembly 26 of the patient table 14. The vertical drive assembly 26 is connected to the T-shaped supporting surface 20 through various mechanisms. For example, in one instance one or more flanges mount the vertical drive assembly 26 to the T-shaped supporting surface 20. The forgoing technique reduces alignment issues relative to a patient table and gantry that are separately mounted to a floor that undulates and does not define an accurate plane of reference.

FIGS. 3-7 illustrate various detector head configurations. FIG. 3 shows a configuration with the detector heads 10 about 180 degrees apart from one another (a 180 degree configuration) and with a patient lying on the pallet 22 extended between the detectors 10. For conventional (non-cardiac) tomograms, the 180 degree configuration is typically used. FIG. 4 illustrates the system with the pallet 22 fully retracted and the patient supported by a device (not shown) other than the pallet 22, such as a transport gurney or a moveable surgical table. With the pallet 22 retracted to the second side 8 of the gantry 2, there are no obstructions on the first side 6 of the gantry 2 that prevent the patient support device (e.g., such as a gurney, a bed, a wheelchair, etc.) to be variously positioned with respect to the detectors 10 to scan the patient.

FIG. 5 illustrates an example of imaging a standing patient. With the patient table 14 located on the second side 8 of the stationary gantry 2 opposite the detectors 10 and the patient pallet 22 is retracted, the patient is suitably positioned for a standing scanning procedure without concern for patient table related pinch point hazards or access limitations. The detectors 10 are rotated by lever or motion control to face one or both the detectors 10 toward the patient (or other objects) outside the ring of the stationary gantry 2. For example, the detectors 10 are attached to side plates (not shown), which are attached to a shaft with bearings (not shown). A retractable lever (not shown) is attached to a detector side plate that connects a plunger (not shown) to slotted features (not shown) on a bearing housing (not shown) that allow it to rotate and lock at various angles (e.g., from about 0 to about 360 degrees). In another instance, the detectors 10 are rotated by a motor, for example, through a motion control system. In this configuration, a motor (not shown) and a gear box (not shown) replace the shaft with bearing, the lever and the plunger, and the user rotates the detectors 10 through motion control software.

FIG. 6 shows a 90 degree configuration with the detector heads 10 about 90 degrees apart from one another and with a patient lying on the pallet 22 extended into between the detectors 10. Cardiac procedures are typically performed with the 90 degree configuration.

It is to be appreciated that with the scanning system described herein the detectors 10 can be positioned at essentially an infinite number of relative angles between about 90 degrees and about 180 degrees. A mechanism to achieve the foregoing is illustrated in FIGS. 2 and 7, which shows a gear motor 28 that turns a gantry rotate gear 30 through a shaft 32 and a pinion 34. One of the detectors 10 is mounted to and rotates with the gear 30. The other detector 10 is mounted to a shaft 36 and positioned within an acruate slot 38 with bearings 40, which permits it to move about 90 degrees of relative rotation. The acruate slot 38 facilitates setting the relative angle between the detectors 10 to establish a 90 to 180 configuration. The detector 10 not mounted to the gear 30 is locked/unlocked from a disc 42 through a (“C” shaped) calliper 44 such that the detectors 10 rotate together via the gear motor 30. When the calliper 44 engages, the disc 42 draws into contact with the detector 10 and the gantry rotate gear 30, and the detector 10 locks to the gear 30. When the calliper 44 disengages, the detector 10 is unlocked from the gear 30 and can freely move independent therefrom. Thus, once a relative angle is set through moving the detector 10, it is locked in place, and the both detectors 10 rotate to scan the patient. In operation, the calliper 44 is released after the detector 10 (attached to the disc 42) is at the bottom of the stationary gantry 2 (6O'Clock). After the calliper 44 disengages, the gantry rotate gear 30 rotates while the head remains at 6 O'Clock, thus bringing the two detector heads 10 to a selected relative angle between about 90 and about 180 degrees as which time the calliper is locked.

FIGS. 8-10 describe the collimator exchange and storage system 24. FIG. 8 provides a top-down view of the patient scanning system. The collimator exchange and storage system 24 includes a delivery track 48 and an extension track 50 that hold at least one pair of carriages 52. The carriages 52 are shown in a storage position on the second side 8 of the stationary gantry 2 and in a loading/unloading position between the detectors 10 on the first side 6 of the stationary gantry 2. Although only a single pair of carriages 52 is illustrated, it is to be appreciated that several pairs are often positioned parallel to the pair on the second side 8 (behind) the gantry 2. Each pair moves from a corresponding slot of the delivery track 48 to the extension track 50, which is extended through the aperture 4 to load/unload collimators from the detectors 10. Both the delivery track 48 and the extension track 50 reside below the pallet 22 (as shown in FIG. 10a), and the extension track 50 extends sufficiently to center transported collimators between the detector heads 10 for mounting/dismounting. The tracks 48 and 50 move up and down with the vertical drive assembly 26. One or more detector radius drives (not shown) allow the detectors 10 to move towards the collimators supported on the extension track 50.

FIG. 9 illustrates the detectors 10 drawn to the collimator mounting/dismounting position. In this position, collimators 54 are easily removed from the detectors 10 to the carriages 52 or loaded from the carriages 52 on the detectors 10. FIG. 10 illustrates a location of the delivery track 48 and the extension track 50 of the collimator exchange and storage system 24 with respect to the patient table 14. The delivery track 48 and the extension track 50 reside below the patient pallet 22 and becomes exposed when the patient pallet 22 is tilted upward. In one embodiment, the delivery track 48 and the extension track 50 are one unit mounted to stationary gantry 2 and patient table 14 such that at least a portion of the unit is within a recess 56 of a supporting structure of the pallet 22. The extension track 50 extends through the recess 56 to a position between the detectors 10. The carriages 52 of the collimator exchange and storage system 24 are maneuvered manually by an operator or, alternatively, through a motion control system.

FIG. 10a illustrates the collimator exchange and storage system 24 and a table drive mechanism 66 looking through the aperture 4 (or bore) from the first side 6 of the stationary gantry 2. The stationary gantry 2 and the rotating gantry 12 are not shown for purposes of clarity. The table drive mechanism 66 includes a motor 68 that engages a belt 70 to rotate a pully 72 to drive the pallet 22 along a lead screw 74 in a longitudinal direction through the aperture 4. The pallet 22 is operatively connected to one or more linear blocks 76 that translate along one or more linear rails 78 affixed to a stationary structure 80 of the patient table 14.

FIGS. 11 and 12 illustrate an exemplary delivery/extension track assembly 58 of the collimator exchange and storage system 24. FIG. 11 shows the delivery track 48 with a plurality of tracks 60. Each slot 60 receives a slide block 62 of a pair of carriages (or gimbals) 52. The extension track 50 has one or more spring safety latches 64 located at the each delivery/extension track junction. The spring safety latches 64 engage and lock when the slide block 62 is positioned within the extension track 50 and the extension track 50 starts to extend. FIG. 12 illustrates the delivery/extension track assembly 58 with the extension track 50 extended and the spring safety latches 64 latched.

FIGS. 13-16 illustrate the delivery of a pair of collimators 54. The carriages 52 via the slide block 62 move along the delivery track 48 to the extension track 50 and pivots into the extension track 50. The extension track 50 extends through the aperture 4 to a position between the detectors 10. In FIG. 13, the carriages 52 with collimators 54 are in a storage position in the delivery track 48. In FIG. 14, the carriages 52 with collimators 54 are moved along one of the tracks 60 to the extension track 50. In FIG. 15, the carriages 52 with collimators 54 are rotated onto the extension track 50, and in FIG. 16, the extension track 50 is extended to position the collimators 54 proximate to the detectors 10 for loading/unloading. After the collimators 54 are mounted to the detector heads 10, the empty carriages 52 are returned to the track 60 of the delivery track 48. To change the collimators 54, the empty carriages 52 move as described above to the position between the detector heads 10 where the collimators 54 are received in the carriages 52. The collimators 54 are released from the detector heads 10 and the heads 10 move apart. The extension track 50 is withdrawn and the carriers 52 are loaded into the corresponding track 60 of the delivery track 48. Then, carriages carrying the next pair of collimators are positioned between the detectors and the collimators are loaded on the detectors as described above.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. An integrated patient scanner system, comprising:
a stationary gantry that has a first side and a second side;
a rotating gantry rotatably mounted to rotate in the stationary gantry around a patient receiving aperture, the rotating gantry defining a patient receiving aperture and
at least one detector mounted to the rotating gantry and extending from the first side around the patient receiving aperture; and
a patient table having:
a pedestal mounted to and extending from the second side of the stationary gantry opposite to the at least one detector such that the pedestal and the stationary gantry define a common T-shaped supporting surface adapted to rest on the floor, and
a patient pallet mounted to the pedestal for movement to extend cantilevered through the patient aperture and out the first side past the at least one detector.

2. The integrated patient scanner system as set forth in claim 1, wherein the patient table includes a vertical drive mounted to the common T-shaped supporting surface.

3. The integrated patient scanner system as set forth in claim 1, wherein the patient table and the stationary gantry include a means for factory calibrating alignment of the stationary gantry and the patient table prior to shipment and installation.

4. The integrated patient scanner system as set forth in claim 1, wherein the patient pallet is mounted for movement along the pedestal to a retracted position in which the pallet is retracted behind the first face such that it does not interfere with an auxiliary patient support positioned adjacent to the least one detector and the first face.

5. The integrated patient scanner system as set forth in claim 1, wherein the first face of the stationary gantry is recessed behind the at least one detector to facilitate performing a procedure with a patient standing or in one of a bed, chair, and a wheel chair.

6. The integrated patient scanner system as set forth in claim 1, further including a second detector, both detectors being configured to detect one of a single photon emission and a positron emission.

7. The integrated patient scanner system as set forth in claim 6, wherein the rotating gantry defines an arcuate slot extending over at least 90 degrees and the second detector includes:
a shaft extending through the arcuate slot;
bearings between the shaft and the rotating gantry to facilitate movement of the shaft along the slot;
a side plate; and
an actuator which selectively locks the shaft and the rotating gantry against movement along this side such that the second detector is selectively positional over 90 degrees.

8. The integrated patient scanner system as set forth in claim 6, further including a collimator exchange and storage system mounted to the back of the stationary gantry adjacent the second side.

9. The integrated patient scanner system as set forth in claim 8, the collimator exchange and storage system includes:
a delivery track that stores collimators; and
an extension track that extends through the aperture of the stationary gantry and provides a path to move the collimators from the delivery track to a position between the detectors.

10. The integrated patient scanner system as set forth in claim 9, wherein the extension track includes at least one safety latch between the delivery track and the extension track to hold the collimators in the extension track when the extension track is extended.

11. A method of manufacturing a patient scanner, comprising:
at a manufacturing site, aligning a pedestal of a patient table which carries a patient pallet with a second side of a stationary gantry which is configured to support a rotating gantry with at least one detector extending from a first side opposite to the second side;
connecting the aligned stationary gantry and the table pedestal in the aligned configuration; and
in an examination room, installing the aligned and connected stationary gantry and the patient table as an integrated unit.

12. The method as set forth in claim 11, wherein the connecting step is performed at the manufacturing site and further including:
shipping the connected stationary gantry and the patient table pedestal as an integral unit from the manufacturing site to the examination room.

13. The method as set forth in claim 11, wherein the aligned patient table and stationary gantry are separated for shipment and reconnected in the examination room.

14. The method as set forth in claim 11, further including:
mounting the detector heads to the rotating gantry with the detector heads cantilevered from the first side; and
mounting a collimator exchange and storage assembly to the second side of the stationary gantry.

15. The method as set forth in claim 14, further including:
sliding a pair of carriages holding a set of collimators from the delivery track through extension track supported by the table pedestal to a position between the detector heads;
raising and lowering the table pedestal to align the collimators and the detector heads;
moving the detector heads together to contact the collimators; and connecting the collimators to the detector heads.

16. A method of imaging a first patient with a patient scanner, comprising:
extending a patient pallet to a cantilevered position on a second side of the stationary gantry through a patient receiving aperture in a rotating gantry, the patient pallet being mounted to a pedestal fixed to the second side of the stationary gantry;
loading a first patient on the patient pallet at a first side of the stationary gantry, the fixed pedestal countering the patient's weight on the cantilevered patient pallet;
positioning the first patient in a scanning field of view with respect to at least one detector head;
rotating the at least one detector head around the first patient; and
imaging the first patient.

17. The method as set forth in claim 16, further including loading a pair of collimators on the first and second detector heads by:
retracting the patient pallet to the second side of the stationary gantry opposite the detector heads;
moving a pair of carriages carrying the collimators from a storage position in a delivery track to an extension track;
extending the extension track with the collimators through the patient receiving aperture to a position between the detectors heads on the first side of a stationary gantry;
moving the detector heads together to contact the set of collimators; connecting the collimators to the detector heads; and
returning the carriages to the storage position.

18. The method as set forth in claim 16, further including unloading a pair of collimators from first and second detector heads by:
retracting the patient pallet to the second side of the gantry;
moving an empty pair of carriages from the storage position in a delivery track to an extension track;
extending the extension track through the patient receiving aperture to position the empty pair of carriages between the detectors heads on the first side of a stationary gantry;
moving the detector heads together, connecting the collimators carried by the detector heads to the carriages, and disconnecting the collimators from the detectors heads;
moving the detector heads apart;
retracting the carriers through the aperture to the second side; and
storing the collimators in the delivery track behind the on the second side of stationary gantry.

19. The method as set forth in claim 16, further including imaging a second patient in a standing, sitting, or transverse prone position, comprising:
removing the first patient from the patient pallet;
retracting the patient pallet to the second side of a stationary gantry opposite the at least one detector head;
positioning the second patient in a scanning field of view with respect to at least one detector head on the first side of the stationary gantry, with the second patient one of standing, sitting, and laying on an ancillary patient support device; and imaging the patient.

20. A patient scanner system, comprising:
a stationary gantry that has a first side and a second side;
a rotating gantry rotatably mounted to rotate in the stationary gantry around a patient receiving aperture,
a first detector mounted to the rotating gantry and extending from the first side around the patient receiving aperture;
a second detector mounted to an arcuate slot extending over at least 90 degrees in the rotating gantry, the second detector includes:
a shaft extending through the arcuate slot;
bearings between the shaft and the rotating gantry to facilitate movement of the shaft along the slot;
a side plate; and
an actuator which selectively locks the shaft and the rotating gantry against movement along this side such that the second detector is selectively positional over the 90 degrees; and
a patient table to position a patient pallet in a scanning position relative to the first and seconds detectors.

21. An patient scanner system, comprising:
a stationary gantry that has a first side and a second side;
a rotating gantry rotatably mounted to rotate in the stationary gantry around a patient receiving aperture, the rotating gantry defining a patient receiving aperture;
at least one detector mounted to the rotating gantry and extending from the first side around the patient receiving aperture;
a patient table having:
a pedestal adapted to rest on the floor, and
a patient pallet mounted to the pedestal for movement to extend cantilevered through the patient aperture and out the first side past the at least one detector; and a collimator exchange and storage system mounted to the back of the stationary gantry adjacent the second side, the collimator exchange and storage system including:
a delivery track that stores collimators; and
an extension track that extends through the aperture of the stationary gantry and provides a path to move the collimators from the delivery track to a position between the detectors.
